SEQUENCE LISTING (4 sequences)

SEQ ID NO: 1 - protein, 42 aa, Homo sapiens
  DOMAIN (1)..(42): KRAB domain
  Note The residues named 1-42 herein correspond to the residues 4-45 of the gene produce coded by RITA or to residues 4-45 of the sequence shown in SEQ ID NO4., respectively
GLVTFADVAIDFSQEEWACLNSAQRDLYWDVMLENYSNLVSL

SEQ ID NO: 2 - protein, 333 aa, Homo sapiens
  DOMAIN (1)..(333): zinc finger domain
  Note The residues named 1-333 herein correspond to residues 131-463 of the gene product coded by RITA or to the sequence shown in SEQ ID NO4, respectively
FECKDCGKAFSRGYQLSQHQKIHTGEKPYECKECKKAFRWGNQLTQHQKIHTGEKPYECKDCGKAFRWGSSLVIHKRIHTGEKPYECKDCGKAFRRGDELTQHQRFHTGEKDYECKDCGKTFSRVYKLIQHKRIHSGEKPYECKDCGKAFICGSSLIQHKRIHTGEKPYECQECGKAFTRVNYLTQHQKIHTGEKPHECKECGKAFRWGSSLVKHERIHTGEKPYKCTECGKAFNCGYHLTQHERIHTGETPYKCKECGKAFIYGSSLVKHERIHTGVKPYGCTECGKSFSHGHQLTQHQKTHSGAKSYECKECGKACNHLNHLREHQRIHNS

SEQ ID NO: 3 - DNA, 2015 bp, Homo sapiens
  CDS (187)..(1575): coding region for RITA
cgcttctgcaccggtgacgcaaccgctgtgtctccgccagtccgcgcaggccagcatccttcagaaaaagcatccccgaggaggaagacgaatcgttaaacatcttaggtcagctctagcctctcggaatttgtcttcttcagtggaaaccccgagaagactgatcagttcttcagttctaaaacaatggcccagggtttggtgacgttcgccgacgtagccatagacttttctcaggaggagtgggcctgtctgaactctgctcagagggacctgtactgggacgtgatgctggagaactacagtaacttggtctcactggatttggagtcagcatatgaaaataagagtttacctacagaaaaaaacattcatgaaataagggcttccaaaaggaattcagatagaagaagtaaatcccttggccgtaactggatatgtgaaggtacgcttgaaagaccacagcgctccagagggaggtatgtcaatcagatgatcatcaattatgtcaaaaggcctgctactagagaaggcacccctcctagaacacatcagagacatcataaggagaattcctttgaatgtaaggactgtgggaaggcctttagtcgtggctatcaacttagtcaacatcagaaaatccatactggtgagaaaccttatgaatgtaaagaatgtaagaaggccttccgttggggcaatcagcttactcaacatcaaaaaattcatactggggagaagccctacgaatgtaaagactgtgggaaggcttttcgatggggctcaagcctcgttattcataagaggattcatactggtgaaaaaccctatgaatgtaaagactgtggaaaggcctttcggcgtggtgatgagctcactcagcaccagagattccacactggggagaaagactacgaatgcaaagactgtgggaagacctttagccgtgtgtataaacttattcagcacaagagaattcatagtggggagaagccttacgagtgtaaagactgtgggaaggcttttatttgtggttcaagcctcattcagcataaaagaattcacacaggtgagaaaccctatgaatgtcaagaatgtgggaaggcctttactcgagtcaattaccttactcagcatcagaagatccacaccggtgagaagcctcacgaatgtaaggagtgtgggaaggcctttcgctggggttcgagcctcgttaagcacgagaggatacatacgggcgagaagccgtacaagtgcacagaatgtgggaaggccttcaattgtggctatcacctcactcagcacgagagaatccacacaggcgaaaccccgtataaatgtaaggagtgtgggaaggctttcatttatggatcgagcctcgtgaaacatgagagaattcataccggggtgaaaccctatgggtgtacagaatgtgggaagagctttagtcacggccatcagcttacacaacatcagaaaacgcacagtggggcgaaatcctacgaatgtaaggagtgcgggaaggcatgtaaccacctaaaccatctccgagaacatcagaggatccacaacagttgaagagccttttgaacgcagtagcccgctcgtatctatggtttcgctttccacagtttgttacctgcagtcaactgcagttcaaaaatattaaatggaaaattccagaaataaagaattttaagtctcaaatggtgtgcccttctgagtagcgtgatgaaatctctcgctgtccggctccagccggccggggatgtgagtcatcccttggtccagcacatccacgctgtatacgccacccaccctgctagtgacttagtagccgtcttggtgatcagatcaactatcccagcatcacagtgcctgtgcccaagtagtcctcactttgcttaacagtggccccagagagcaggagtagtgatgctggtgattcggatatgccaaagagaagccacaaagtgcttccttttaaatgaaaaggtgaaagttctcaacttaaa

SEQ ID NO: 4 - protein, 463 aa, Homo sapiens
MAQGLVTFADVAIDFSQEEWACLNSAQRDLYWDVMLENYSNLVSLDLESAYENKSLPTEKNIHEIRASKRNSDRRSKSLGRNWICEGTLERPQRSRGRYVNQMIINYVKRPATREGTPPRTHQRHHKENSFECKDCGKAFSRGYQLSQHQKIHTGEKPYECKECKKAFRWGNQLTQHQKIHTGEKPYECKDCGKAFRWGSSLVIHKRIHTGEKPYECKDCGKAFRRGDELTQHQRFHTGEKDYECKDCGKTFSRVYKLIQHKRIHSGEKPYECKDCGKAFICGSSLIQHKRIHTGEKPYECQECGKAFTRVNYLTQHQKIHTGEKPHECKECGKAFRWGSSLVKHERIHTGEKPYKCTECGKAFNCGYHLTQHERIHTGETPYKCKECGKAFIYGSSLVKHERIHTGVKPYGCTECGKSFSHGHQLTQHQKTHSGAKSYECKECGKACNHLNHLREHQRIHNS

(19) United States
(12) Patent Application Publication
(10) Pub. No.: US 2007/0020275 A1
(43) Pub. Date: Jan. 25, 2007

(54) Nucleic acid sequences of hyperplasia and tumours of the thyroid

(76) Inventors: Jorn Bullerdiek, Bremen (DE); Volkhard Rippe, Emtinghausen (DE); Maren Meiboom, Bremen (DE); Gazanfer Belge, Bremen (DE)

Correspondence Address: WOOD, PHILLIPS, KATZ, CLARK & MORTIMER, 500 W. MADISON STREET, SUITE 3800, CHICAGO, IL, 60661, US

(21) Appl. No.: 11/504,392

(22) Filed: Aug. 15, 2006

Related U.S. Application Data
(63) Continuation of application No. 10/111,257, filed on Apr. 11, 2002, now abandoned.

(30) Foreign Application Priority Data
Dec. 10, 1999 (DE) DE 19949179.8

(51) Int. Cl. C12Q 1/68; G01N 33/574; C07H 21/04; C12P 21/06; A61K 39/395; C07K 14/82; C07K 16/30

(52) U.S. Cl. 424/155.1; 435/6; 435/7.23; 435/69.1; 435/320.1; 435/325; 530/350; 530/388.8; 536/23.5; 514/44

(57) ABSTRACT

The invention relates to a nucleic acid which codes for a polypeptide. Said polypeptide comprises a KRAB domain and/or at least one zinc finger motif, whereby the KRAB domain contains the amino acid sequence of SEQ ID NO: 1 and the zinc finger motif is of the type contained in the zinc finger domain with the amino acid sequence of SEQ ID NO: 2.

The present invention is related to a nucleic acid which codes for a polypeptide, said polypeptide comprising a KRAB domain and/or at least one zinc finger motif. The invention is further related to vectors, polypeptides, cells, antibodies, ribozymes, their use to diagnose and/or treat functional disorders of the thyroid and/or hyperplasia of the thyroid and/or thyroid tumours and their use to produce a medicament, pharmaceutical compositions and kits, as well as methods for detecting functional disorders of the thyroid, hyperplasia of the thyroid and/or tumours of the thyroid, primers for preparing the nucleic acid, methods for preparing the nucleic acid and a pharmaceutical composition.

It has been known for a long time in medicine that the hormone production of the thyroid is important for the control of growth and development of the body and the changes associated with a functional disorder of this gland that are at least in some cases conspicuous in the true sense of the word are also well known.

With regard to the pathogenesis of strumae and thyroid tumours in general it has not yet been possible to develop a comprehensive concept especially at the molecular level. At present two concepts are being discussed one of which assumes that hyperplastic tissue is a result of chronic stimulation by a trophic hormone which finally leads to the growth of polyclonal nodules. This concept is referred to as non- neoplastic endocrine hyperplasia (NNEH). The second concept assumes that the nodules are real clonal tumours.

In the case of the thyroid it has turned out that the simple concept that applies to other glands cannot be applied to non-neoplastic endocrine hyperplasia. A review of the current concepts in this field may be found in Studer, H. (1995); Endocrine Reviews, vol. 16. no. 4. pages 411-426.

Hence an early diagnosis is necessary for the treatment of strumae and thyroid tumours in order to allow the use of suitable therapeutic concepts.

The object of the present invention was to provide agents for diagnosing and treating functional disorders of the thyroid, hyperplasia of the thyroid and tumours of the thyroid at the molecular level, and in particular it is intended to provide nucleic acid sequences which are involved in the pathogenicity mechanisms and are suitable for examining these in more depth as well as to provide sequences which have an effect on or can influence the pathogenicity mechanisms.

Furthermore drugs which are based thereon and general pharmaceutical compositions should be provided.

The same also applies to polypeptides derived from the nucleic acid sequences and antibodies and ribozynies directed against them and pharmaceutical compositions which contain them.

Furthermore it is intended to provide kits for diagnosing and/or treating functional disorders of the thyroid, hyperplasia of the thyroid and tumours of the thyroid as well as methods for their detection.

The object is achieved according to the invention by a nucleic acid which codes for a polypeptide, wherein the polypeptide comprises a KRAB domain and/or at least one zinc finger motif and the KRAB domain comprises the amino acid sequence of SEQ ID NO: 1 and the zinc finger motif is one which is contained in the zinc finger domain having the amino acid sequence of SEQ ID NO:2.

The object is also achieved by a vector which contains the nucleic acid sequence according to the invention.

Finally the object is achieved by a polypeptide which comprises a KRAB domain and/or a zinc finger domain and is coded by a nucleic acid sequence according to the invention.

Furthermore the object is achieved by a cell which contains a vector according to the invention.

The object is additionally achieved by an antibody which is directed against a polypeptide according to the invention.

Moreover the object is achieved according to the invention by an antibody which is directed against a nucleic acid sequence according to the invention.

The object is additionally achieved according to the invention by a ribozyie which is directed against a nucleic acid sequence according to the invention.

In addition the object of the invention to provide medicaments and pharmaceutical compositions comprising such agents and in particular for the diagnosis and/or treatment of functional disorders of the thyroid, hyperplasia of the thyroid and/or tumours of the thyroid, is achieved by a nucleic acid according to the invention or its use, by a vector according to the invention or its use, by a polypeptide according to the invention or its use, by a cell according to the invention or its use, by an antibody according to the invention or its use, by a ribozyme according to the invention or its use and by a pharmaceutical composition which contains an agent which is selected from the group comprising a nucleic acid, a vector, a polypeptide, a cell, an antibody, a ribozyme, each of which are according to the invention, and combinations thereof and a pharmaceutically acceptable carrier.

The object is also achieved by a kit according to the invention for diagnosing functional disorders, hyperplasia and tumours of the thyroid, wherein the kit contains at least one element which is selected from the group comprising a nucleic acid, a vector, a polypeptide, a cell, an antibody and a ribozyme, each according to the invention.

The method according to the invention for detecting functional disorders, hyperplasia and/or tumours of the thyroid which comprises the steps contacting the thyroid material with an agent which is selected from the group comprising a nucleic acid sequence, a vector, a polypeptide, an antibody and a ribozyme each according to the present invention and determining whether functional disorders, hyperplasia and/or tumours are present also achieves the object according to the invention.

The object is furthermore achieved by using a nucleic acid sequence according to the invention as a primer.

Furthermore the object is achieved according to the invention by a primer for preparing and/or screening and/or detecting a nucleic acid which is complementary to a part of a nucleic acid sequence according to the invention. The sequence of this primer can also be derived by mutation or labelling of a nucleic acid sequence according to the invention.

The object is also achieved according to the invention by a method for preparing a nucleic acid which contains a sequence that can be detected in thyroid tumours in which a translocation with a breakage point is present in the chromosomal band 19q13, the said sequence being located within the chromosomal band 19q13, characterized in that the method comprises the steps:
preparing primers according to the invention for carrying out a polymerase chain reaction,
preparing a nucleic acid sequence taken from the band 19q13 of the human chromosome 19 or a nucleic acid according to the invention,
mixing the nucleic acid sequence or the nucleic acid with the primers,
carrying out a polymerase chain reaction.

One embodiment provides that the nucleic acid according to the invention comprises the nucleic acid sequence of SEQ ID NO: 3.

Another embodiment provides that the nucleic acid according to the invention can hybridize to the nucleic acid sequence of SEQ ID NO:3 and/or the nucleic acid sequence which codes for a polypeptide, said polypeptide comprising a KRAB domain and/or a zinc finger domain and the KRAB domain comprising the amino acid sequence of SEQ ID NO: 1 and the zinc finger domain comprising the amino acid sequence of SEQ ED NO: 2.

One embodiment provides that the nucleic acid according to the invention has at least one mutation.

One embodiment provides that the nucleic acid according to the invention is in the form of an antisense nucleic acid for one of the sequences according to the invention.

Furthermore provision can be made for the nucleic acid according to the invention to be modified.

The vector according to the invention can additionally contain one element which is selected from the group comprising promoters, terminators and enhancers.

A preferred embodiment provides that the vector is an expression vector.

A further preferred embodiment provides that at least one promoter is in-frame with a part of the nucleic acid sequence(s) according to the invention coding for a polypeptide.

A particularly preferred embodiment provides that the promoter is a eukaryotic promoter.

Furthermore the vector can additionally contain an additional nucleic acid sequence which is in the reading frame together with a part of a nucleic acid sequence according to the invention coding for a polypeptide, wherein the additional nucleic acid sequence is selected from the group of nucleic acid sequences comprising signal sequences and sequences which code for domains of proteins.

The polypeptides according to the invention can be modified.

The polypeptide according to the invention can also be derived from an amino acid sequence by conservative or silent amino acid mutation.

In the case of the cell according to the invention it can be provided that a nucleic acid sequence according to the invention is integrated into the genome of the cell.

In a further embodiment of the cell according to the invention it can be provided that a nucleic acid sequence according to the invention is present integrated into the human chromosome 19 and in particular into band 19q13.

Furthermore the cell which is at least diploid with regard to human chromosome 19 can be such that it only has one chromosome containing a nucleic acid sequence according to the invention.

A particularly preferred embodiment provides that the cell according to the invention is a eukaryotic cell.

The antibodies according to the invention can be monoclonal antibodies.

A further embodiment can provide that the antibodies according to the invention are modified.

In one embodiment the ribozyme according to the invention comprises at least a part of a nucleic acid sequence according to the invention.

A vector according to the invention and/or a cell according to the invention and/or a ribozyme according to the invention can be used to diagnose and/or treat functional disorders of the thyroid and/or hyperplasia of the thyroid and/or thyroid tumours.

A vector according to the invention and/or a cell according to the invention and/or a ribozyme according to the invention can be used to prepare a medicinal drug.

When the kit according to the invention for diagnosing and/or treating functional disorders, hyperplasia and tumours of the thyroid is in particular used for diagnostic applications it can comprise one of the nucleic acid sequences according to the invention and/or an antibody according to the invention and especially one which is directed against the polypeptides according to the invention.

A further embodiment provides that the kit contains at least one cell according to the invention.

Furthermore the invention proposes that when the kit according to the invention is used for therapeutic applications, it contains a nucleic acid sequence, a polypeptide, an antibody and/or a ribozyme each according to the present invention.

The method according to the invention for detecting functional disorders, hyperplasia and/or tumours of the thyroid can be such that the thyroid material is present ex vivo.

In one embodiment of the use according to the invention of a nucleic acid according to the invention it can be provided that this nucleic acid is used as a primer. In this case a preferred embodiment is that, starting with a strand of a nucleic acid according to the invention, a nucleic acid sequence is determined that can hybridize with a part of the nucleic acid according to the invention and that this nucleic acid sequence is synthesized. Furthermore another preferred embodiment provides that starting with another strand which is complementary to the previously used strand, a nucleic acid sequence is selected which can hybridize with a part of this complementary strand and that this nucleic acid sequence is synthesized.

In the following tumours refers to malignant as well as benign tumours.

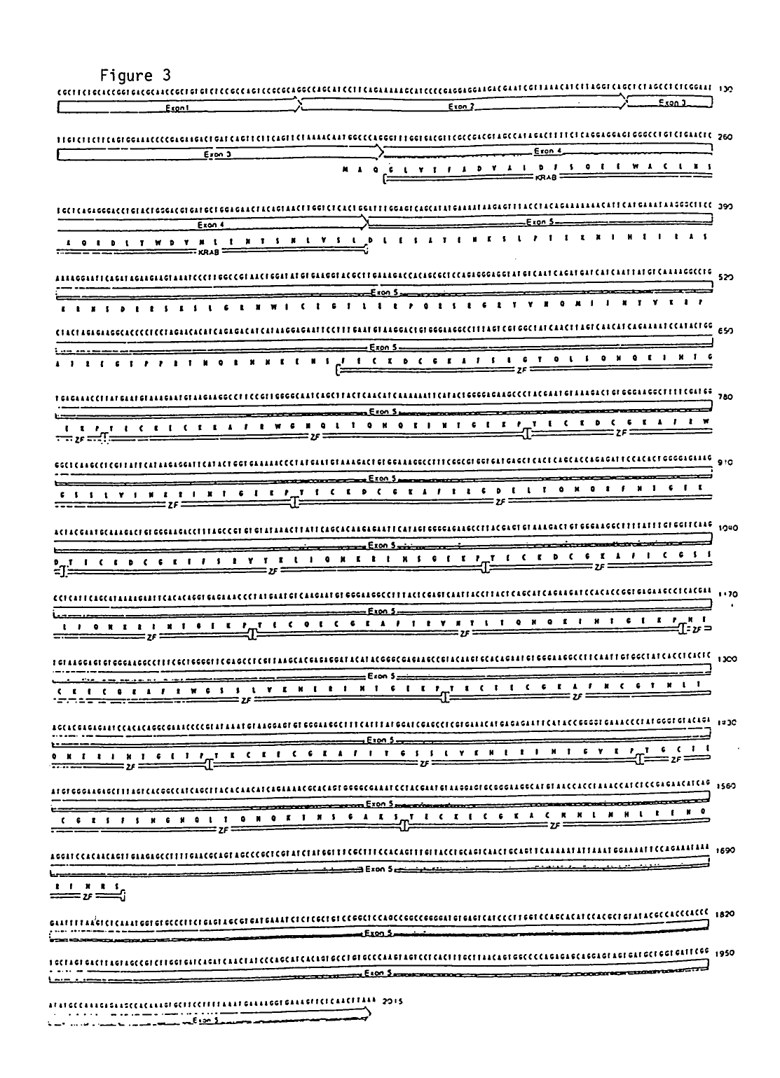

The basis of the invention was the surprising finding that the chromosome translocations on the human chromosome 19 observed in tissue specimens from follicular thyroid tumours, including thyroid carcinomas and strumae involve the chromosomal band 19q13 and that in particular all breakage points can be allocated to a particular nucleic acid sequence within band l9q13 as disclosed herein. In particular the inventive nucleic acids disclosed herein appear to be target nucleic acids for the aberrations 19q13 and in particular in epithelial tumours. In this connection target nucleic acid means that the observed chromosomal aberrations occur in the nucleic acid according to the invention or in its immediate vicinity. In this connection immediate vicinity means within a distance of about 200 kbp. The 3′ end of the nucleic acid according to the invention as well as the 3′ end of the cDNA shown in FIG. 3 (SEQ ID NO: 3) can be a reference point for this.

Furthermore it was surprisingly found that it is possible to produce cells which have the chromosomal change i.e. the chromosome translocations in band 19q13 as shown in example 2.

Finally it was also surprisingly found that the sequence within which the breakage points of the chromosome translocations are located, is expressed in thyroid tissue and in particular in thyroid tumour tissue.

In connection with the present invention zinc finger domain means a zinc finger motif and in particular a collection of at least two zinc finger motifs although each zinc finger motif per se satisfies the criterium of a domain i.e. the amino acid sequence of the zinc finger motif can already fold as such into a stable tertiary structure. In particular it should be noted in connection with the present invention that the structure referred to as the zinc finger domain contains 12 zinc finger motifs. The exact location of these zinc finger motifs is also shown in FIG. 3 (where the individual zinc finger motifs are labelled with ZF). In addition example 12 shows the exact nucleotide borders for the individual zinc finger motifs.

According to the present invention the nucleic acid according to the invention can be in various forms in the different embodiments. The following nucleic acids are listed purely for the purposes of illustration. One embodiment comprises the nucleic acid which codes for a KRAB domain of the amino acid sequence shown in SEQ ID NO: 1. Another embodiment of the nucleic acid according to the invention codes for a zinc finger motif as contained in the amino acid sequence of SEQ ID NO:2. More precisely such a nucleic acid sequence can contain one or more of the nucleic acid sequences that code for an amino acid sequence that is coded by a nucleic acid sequence of nucleotide positions 577 -660, 661 -744, 745 -828, 829 -912, 913 - 996, 997-1080, 1081 -1164, 1165 -1248, 1249-1332, 1333 -1416, 1417-1500 or 1501 -1575 (as also shown in example 12) of FIG. 3. In other words the nucleic acid according to the invention can contain the nucleic acid from position 577 to 660 or other sequences defined by the aforementioned nucleotide positions. Furthermore it also encompasses those sequences that code for the amino acid sequences defined by the above-mentioned nucleic acid positions and which differ from the nucleic acid sequence shown in FIG. 3 as a result of the degeneracy of the genetic code. Finally the invention also encompasses those nucleic acid sequences which contain the nucleic acid sequence coding for the KRAB sequence of SEQ ID NO: 1 and the nucleic acid sequence corresponding to sequence positions 577 to 660 (the first zinc finger motif of the cDNA shown in FIG. 3 and hence the gene RITA). If the nucleic acid coding for the KRAB domain according to SEQ ID NO: 1 is referred to as A and the nucleic acids coding for the 12 zinc finger motifs shown in FIG. 3 are referred to as B1, B2 etc. to B12, then the nucleic acid according to the invention especially includes those combinations of A with at least one element from the group comprising B1 to B12. In this connection the group formed from the elements B1 to B12 can also contain elements which are in turn combinations of at least two of the elements Bl to B12.

Hence the present nucleic acid sequences allow new approaches to diagnose and to treat or examine mechanisms associated with the occurrence of strumae and thyroid tumours or thyroid carcinomas. In particular knowledge of the sequence allows the direct or indirect use of diagnostic or therapeutic agents at a molecular level in the form of pharmaceutical compositions and drugs.

The nucleic acid sequences according to the invention can be used to design suitable diagnostic and therapeutic agents in the above sense as well as kits and methods in a manner known to a person skilled in this field.

Nucleic acid in this connection means DNA sequences as well as RNA sequences including hybrids thereof and also derivatives thereof with a modified backbone. It also includes single-stranded and double-stranded nucleic acids as well as those with a triple structure.

The strandedness of the DNA i.e. for example whether it is present in a single- stranded or double-stranded form, can change over the length of the nucleic acid sequence.

In particular the nucleic acid sequence according to the invention can also be present as a fragment rather than a complete sequence.

Furthermore within the scope of the present disclosure it is possible for the nucleic acid sequences to be present in a mutated form. Mutations as used herein means all mutations known to a person skilled in that art that can occur within a nucleic acid sequence including point mutations as well as non-point mutations i.e. inversions, insertions and deletions.

The criterion hybridization which is generally recognized in the prior art should be used herein especially with regard to the interaction of the nucleic acid according to the invention with other nucleic acids. It is known that the stringency of hybridization can be changed within a particular range by selecting suitable hybridization conditions and thus enable the nucleic acid sequences according to the invention to hybridize to nucleic acid sequences which can differ by varying degrees from the completely corresponding i.e. complementary sequences.

Nucleic acid sequence in the sense of the invention is also intended to mean any sequence that would hybridize with sequences according to the invention if it were not for the degeneracy of the genetic code. With regard to the open reading frame or the open reading frames present in the nucleic acid sequence according to the invention this means that it/they can be translated into an amino acid sequence using the genetic code. However, as a consequence of the degeneracy of the genetic code it is possible to obtain a nucleic acid sequence from an amino acid sequence obtained in this way again using the genetic code which is so different that it may no longer be able to hybridize with the nucleic acid sequence used to determine the amino acid sequence.

Starting from the nucleic acids disclosed herein it is possible for a person skilled in the art to produce a suitable antisense nucleic acid, in particular antisense RNA that can interact with the nucleic acid sequences according to the invention. This interaction can be the basis for directly influencing the processes involving the nucleic acid sequences. This interaction can for example occur at the level of transcription as well as at the level of translation.

Nucleic acid sequences are generally understood herein as nucleic acid sequences that can be isolated from the said tissue in situ or ex vivo for example from appropriate cell, tissue or organ cultures. However, it also refers to corresponding nucleic acid sequences that can be isolated from gene banks in particular human gene banks and more preferably gene banks of the human chromosome 19. Furthermore the term nucleic acid sequences as used herein should also encompass those that can be produced by suitable synthetic techniques including the polymerase chain reaction and other biochemical and chemical synthetic processes known in the prior art.

In addition the sequences according to the invention can also be present in a modified form.

Modification is understood herein to include fragmentation, insertion, deletion and inversion of (partial) sequences of the nucleic acid sequences according to the invention. It also includes the insertion of other nucleic acid sequences. These nucleic acid sequences can for example code for particular domains, serve as spacers or serve as elements for translation and transcription regulation.

Furthermore the nucleic acids according to the invention can be modified such that they contain sequences or molecules that allow an interaction with other molecules. This can for example be in the form of a binding site on a solid support or a sequence that mediates binding to a nucleic acid binding protein.

In addition the nucleic acid sequences according to the invention can be labelled. Labelling is basically understood herein as direct as well as indirect labelling.

Labelling can be carried out using labels and labelling methods known in the prior art and includes radioactive, non-radioactive and fluorescent labels. Non-radioactive labels include among others digoxigenin, avidin, streptavidin and biotin.

A vector as used herein means in particular recombinant vectors that are known in the prior art. Such vectors include, among others, viral vectors such as adenoviral or retroviral vectors and phage systems, as well as plasmid vectors including cosmid vectors and artificial chromosomes that can be used in prokaryotic and/or eukaryotic systems.

In addition to the nucleic acid sequences according to the invention, the vectors according to the invention can contain additional elements that are known in the prior art. The respective elements such as promoters, terminators and enhancers are selected according to the respective host cell system in a manner known to a person skilled in the art. In particular this means the selection of a suitable eukaryotic promoter and of an inducible promoter. Apart from the said elements it is also possible that such vectors contain elements that result in the integration of at least the nucleic acid sequence according to the invention or a part thereof into the genome of the host cell system.

It is also possible that at least one of the said elements is connected in-frame with at least one open reading frame of the nucleic acid sequences according to the invention and it is especially advantageous when the transcription rate of the special open reading frame is regulated by an additionally inserted promoter in which case the promoter is then typically at a suitable distance to and in-frame with the open reading frame.

Furthermore provision can be made for an open reading frame of the nucleic acid sequences according to the invention to have a signal sequence preferably under the aforementioned conditions which allows a translocation of the gene product coded by the open reading frame which optionally allows a further modification of the gene product. Such signal sequences include those for the transport of the synthesized protein to the endoplasmic reticulum, Golgi apparatus, to lysosomes, to cell organelles such as mitochondria and chloroplasts and to the cell nucleus. Such a passage through various cell compartments allows a post-translational modification and hence optionally a further advantageous structure of the gene product.

Apart from signal sequences, such a construct can also contain additional nucleic acid sequences which have the effect that the gene product of an open reading frame of the nucleic acid sequences according to the invention form a fusion product where the fused part can correspond to a domain of another protein and for example facilitate the detection of the gene product of the open reading frame of the sequences according to the invention or interaction with other molecules or structures in the biological system where the biological system is preferably the cell.

In addition to the aforementioned advantages of the nucleic acid sequences according to the invention and other advantages that a person skilled in the art can derive from the description, these are also inherent to the polypeptides derived from the said nucleic acid sequences. These polypeptides can be on the one hand derived directly from an open reading frame of the nucleic acid sequence according to the invention or they can be produced by a vector that is expressed in a host organism in a manner known to a person skilled in the art. In this case the host organism is typically firstly transformed with the vector according to the invention, the host organism is multiplied and the polypeptide is isolated from the host organism or from the medium if it is secreted into the medium.

In addition to a direct use of the polypeptides according to the invention to influence cellular processes in thyroid tissue, they can also be used to purify other components involved in the cellular processes for example by means of affinity chromatography or to produce suitable antibodies which can then in turn be used, among others, for therapeutic and/or diagnostic purposes.

Depending on the respective requirements with regard to post-translational modification or the desired degree of purity etc., either a prokaryotic or a eukaryotic host organism may be advantageous for producing the polypeptide according to the invention in a host organism.

The polypeptide according to the invention can be modified in a suitable manner. Modification is understood among others as a fragmentation and in particular as a shortening of the molecule. Modification in the sense used herein also includes the labelling of the polypeptide. The latter can be achieved by high molecular as well as low molecular compounds and includes radioactive, non-radioactive and fluorescent labelling. A label can for example also be present in the form of a phosphorylation or glycosylation of the protein. Suitable labelling methods and modification methods are known in the prior art ( see e.g. Protein Methods, 2nd ed. D.M. Bollag et al., Wiley Liss, Inc., New York, N.Y., 1996) and are incorporated herein in their entirety.

Modification according to the invention also means any form of post-translational modification known to persons skilled in the art, in particular proteolytic processing of the polypeptide, attachment or separation of residues at the N-terminus, acetylation of the N-terminus, myristoylation of the N-terminus, attachment of glycosyl phosphatidyl residues, farnesyl residues or geranylgeranyl residues at the C- terminus, N-glycosylation, O-glycosylation, attachment of glycosaminoglycan, hydroxylation, phosphorylation, ADP-ribosylation and formation of disulfide bridges.

It is also possible for the polypeptide according to the invention to be derived from a polypeptide according to the invention by amino acid mutation. Amino acid mutation means a mutation in which an amino acid is substituted for an amino acid with a similar side chain (conservative mutation) for example I by L or D by E as well as a mutation in which an amino acid is substituted by another amino acid without this substitution having a disadvantageous effect on the function of the coded polypeptide (silent mutation). The function of the coded polypeptide can be checked by a suitable assay. Such a functional assay for the KRAB domain and the zinc finger domain is for example a bandshift assay. Useful functional assays are disclosed in Margolin et al., 1994, Proc. Natl. Acad. Sci. USA 91:4509-4513; Witzgall et al., 1994, Proc. Natl. Acad. Sci. USA 91:4514-4518, Andrews, N. C. and Faller, D. V. 1991, Nucleic Acids Res 19(9): 2499, Elser, B. et al., 1997, J. Biol. Chem. 272(44): 27908-12, Fan, C. M. and Maniatis, T., 1989, Embo J. 8(1): 101-110, Gamer, M. M. and Revzin, A. 1981, Nucleic.Acids Res 9(13):3047-3060, Kadonaga, J.T. et al., 1987, Cell 51(6):1079- 1090, Pierrou, S. et al., 1995, Anal. Biochem. 229(l):99-105, Singh, H. et al., 1986, Nature 319(6049): 154-158, Thiesen, H. J. and Bach, C., 1993, Ann N Y Acad. Sci. 684:246-249 and Vissing et al., 1995, FEBS 369:153-147 and are hereby incorporated by reference.

A polypeptide according to the invention can contain a KRAB domain or a zinc finger domain or a combination of both domains.

A polypeptide according to the invention can in particular contain the sequence shown in SEQ ID NO: 4.

The cells according to the invention can be used to yield additional advantages. These include among others the production of corresponding nucleic acid sequences and gene products derived therefrom.

In particular insertion of the nucleic acid sequences according to the invention into the genome of a cell is particularly important for example in order to further study the influence of such sequences especially in the cellular field. In this connection gene-dose effects or such like can be investigated and utilized for diagnostic and/or therapeutic purposes. In this connection a state appears to be especially advantageous in which, starting from diploid cells, only one chromosome contains the nucleic acid sequence according to the invention which is preferably inserted at its corresponding position in band 19q13 and the second chromosome has no chromosome translocation affecting the chromosomal band 19ql3. Such cells can for example be used as positive and/or negative controls in a diagnostic method.

In view of the technical teaching disclosed herein it is then also possible to produce antibodies against the gene product of the nucleic acid sequences according to the invention and also against the nucleic acid sequence itself. This also results in the advantages known to a person skilled in the art which derive from the presence of antibodies against chemical compounds. For example this would enable a purification or detection of the said compounds and also make it possible to influence the biological activity and bioavailability of the compounds against which the antibody is directed in situ as well as ex vivo or in vitro. More precisely monoclonal antibodies can be used to specifically detect gene products or to influence. at a cellular level an interaction of the gene products or nucleic acid sequence with other cellular components and thus to specifically influence the cellular processes. Depending on the effect of the respective compound against which the antibody is directed and how it effects the system in question, it is possible in principle to achieve stimulating as well as inhibiting effects.

Antibodies are understood herein to include polyclonal antibodies as well as monoclonal antibodies. Monoclonal antibodies are, however, particularly preferred due to their increased specificity. However, application cases are conceivable in which the purity and specificity of polyclonal antibodies is sufficient or in which the many other properties and specificities of polyclonal antibodies can be used in an advantageous manner. The production and use of antibodies is described in: A Laboratory Manual (E. Harlow & D. Lane, Cold Spring Harbor Laboratory, N.Y., 1988) and is incorporated herein.

Within the scope of this invention it is also possible for the antibodies to be single- chain antibodies.

The antibodies can also be fragmented and in particular shortened within the scope of the present invention. This includes a substantial truncation of the antibodies provided that the specific property of the antibody is retained i.e. binding to a defined epitope. Truncation is particularly advantageous when the corresponding antibody is to be used at a cellular level since it has improved permeation and diffusion properties compared to a complete antibody.

Furthermore other forms of modification are envisaged which are known to a person skilled in the field and are described for example in Antibodies: A Laboratory Manual (E. Harlow & D. Lane, Cold Spring Harbor Laboratory, N.Y., 1988). In general it can be stated that antibodies can in principle be modified in a similar manner to polypeptides and, to a certain extent, to nucleic acids and that the afore- mentioned also applies in the same sense to the antibodies according to the invention.

With regard to a pharmaceutical composition which contains a ribozyme in addition to a pharmaceutically acceptable carrier or the use of a ribozyme according to the invention as a drug and in particular to treat functional disorders, hyperplasia and tumours of the thyroid, it is also possible for the ribozyme to be constructed such that it specifically acts on the nucleic acid sequences according to the invention and hence in this case regulates expression and translation at a cellular level which is particularly important for the therapeutic and diagnostic aspect. Due to the fact that ribozymes have intramolecular as well as intermolecular catalytic effects, the nucleic acid sequences according to the invention can be changed such that they have regions which are themselves cleaved by the ribozyme activity of the changed region. In particular this also allows therapeutic methods that can be designed by a person skilled in the art in the light of the sequence information that is now available.

Like the nucleic acid sequences according to the invention themselves, ribozymes are of particular advantage when, but not only when, they are introduced in the effector cell for example by gene therapy. However, it is also conceivable that appropriate modifications are carried out ex vivo and such modified cells are then made available for reimplantation which is either allogenic or autogenic. The production and use of ribozymes is disclosed in Ribozyrne Protocols (Philip C. Turner, Ed. Humana Press, Totowa, N.Y., 1997) and is incorporated herein.

With regard to the design of the kit for diagnosing and/or treating functional disorders, hyperplasia and tumours of the thyroid it should be noted that the exact design of such a kit i.e. which specific components are contained therein are known to a person skilled in this field and in particular are possible in the light of the above- mentioned description of the effects and uses of the nucleic acid sequences disclosed herein. The kit according to the invention in any case contains at least one of the elements according to the invention which is selected from the group comprising the nucleic acid sequence, the vector, the polypeptide, the cell, the antibody and the ribozyme each in its inventive form. With regard to the use of the cells according to the invention they can in particular be used as a reimplant or as a positive and/or negative control in corresponding diagnostic or therapeutic methods.

In the method according to the invention for detecting functional disorders and hyperplasia and/or tumours of the thyroid also in the form of carcinomas and strumae, it is possible for the thyroid material to be present in situ, ex vivo or in vitro. The term“thyroid material” as used herein refers to material and in particular cellular material of the thyroid in its normal as well as in a pathological state. Hence the term thyroid material also includes material from thyroid glands with a functional disorder, from hyperplasia of the thyroid, from tumours of the thyroid, including carcinomas and strumae. The determination whether a functional disorder, hyperplasia and/or tumour is present is finally carried out by comparing the effect of the agent or agents according to the invention on the thyroid material to be examined with its/their effect on normal thyroid tissue. Other embodiments of the method according to the invention can be derived by persons skilled in the art on the basis of the disclosure contained herein.

The statements made above with regard to the various embodiments and to the achievable advantages also apply synonymously to the dnigs according to the invention and in particular to those for diagnosing and/or treating functional disorders, hyperplasia and/or tumours of the thyroid in the form of the sequence according to the invention or its use, in the form of a vector according to the invention or its use, in the form of the polypeptide according to the invention or its use, in the form of the cell according to the invention or its use, in the form of the antibody according to the invention or its use and in the formn of the ribozyme according to the invention or its use which are disclosed herein. The corresponding drugs can contain a quantity of one or several of the aforementioned agents.

The same also applies to pharmaceutical compositions according to the invention. In this case the pharmaceutical composition may contain only one of the compounds listed above in addition to the pharmaceutically acceptable carrier. Within the scope of the invention the pharmaceutical composition can also contain several of the said compounds in addition to the pharmaceutically acceptable carrier.

A pharmaceutically acceptable carrier is understood herein as all carriers known to a person skilled in the art that are typically selected depending on the form of administration. A pharmaceutically acceptable carrier can among others be water, buffer solutions, alcoholic solutions and such like.

With regard to the method according to the invention for constricting primers it should be noted that they are those which allow a specific preparation of the nucleic acids according to the invention or parts thereof.

The method according to the invention for preparing a nucleic acid sequence according to the invention can utilize, from which ever source, the nucleic acid sequences according to the invention or corresponding sequences using primers according to the invention as primers for a polymerase chain reaction in all its various embodiments. The design of suitable primers and the procedure for polymerase chain reactions is disclosed in PCR & PCR 2: A practical approach (M.J. McPherson, P. Quirke and G.R. Taylor, ed. IRL Press, Oxford, England 1991, & M.J. McPherson and B.D. Hamers, ed. IkL Press, Oxford, England 1995) and is incorporated herein.

Further features and advantages of the invention derive from the figures, the sequence protocol and the following examples.

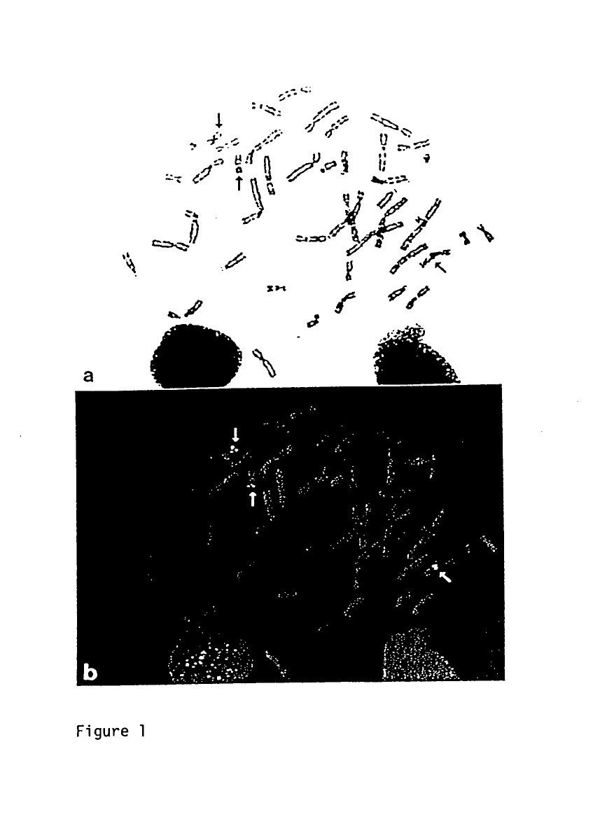

FIG. 1a: shows a metaphase of the cell line S-121T/SV40 with t(5;19)(q13:q13) after G band staining; chromosome 19, derivative chromosome 19 (=“der(19))” and derivative chromosome 5 (=“der(5)” are marked with arrows.

FIG. 1b: shows the same metaphase after FISH with the PAC clone 19/B; the hybridization signals are located on chromosome 19, der(19) and der(5);

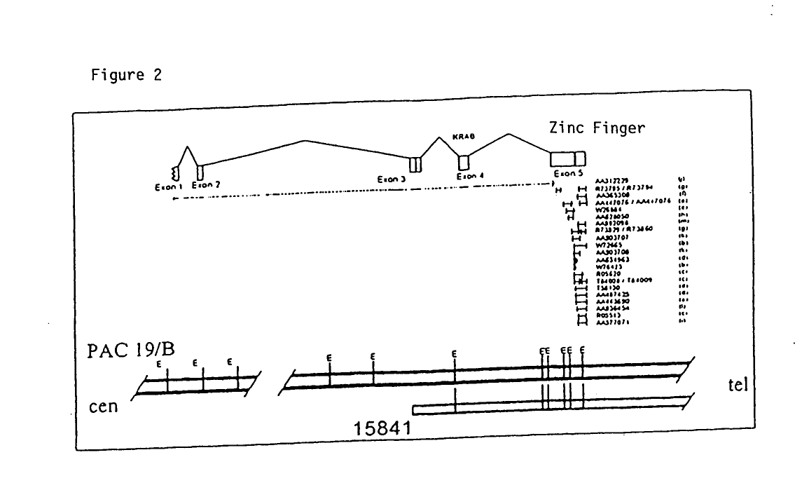

FIG. 2: shows the genomic structure of a gene which is referred to in the following as RITA (rearranged in thyroid adenomas) the gene that is involved in specific 19q13 translocations in thyroid tumours . Grey boxes represent the coding regions which belong to the open reading frame of exon 3 to exon 5, whereas 5′ or 3′ non-translated regions are shown as white boxes. The arrows under the exon-intron structure represents cDNA clones from the data banks. The tips of the arrows show the sequencing direction for these clones. The source of the RNA/cDNA is indicated by a single letter in brackets behind the gene bank accession number of each clone (a: total foetus, b: foetal heart, c: foetal liver/spleen, d: lung, e: eye retina, f: epiphysis, g: breast, h: ovarial-cortical stroma; i: milk duct breast tumours, j: Jurkat T cells, k: germ cell tumour, 1: neuroendocrine lung carcinoma, m: testis). Filled bars in the lower part of the figure represent the position of parts of cosmid 15841 and PAC19/B relative to RITA; the abbreviations “cen” and “tel” relate to the position of the centromer and the telomer relative to RITA; the orientation of the gene is from left to right in the figure in the direction of the telomer.

FIG. 3: shows the cDNA sequence of RITA and also shows the size of the exons, the open reading frame and the amino acid sequence of the open reading frame derived therefrom. The regions corresponding to the KRAB domain and the zinc finger motifs (ZF) are indicated by bars with black borders;

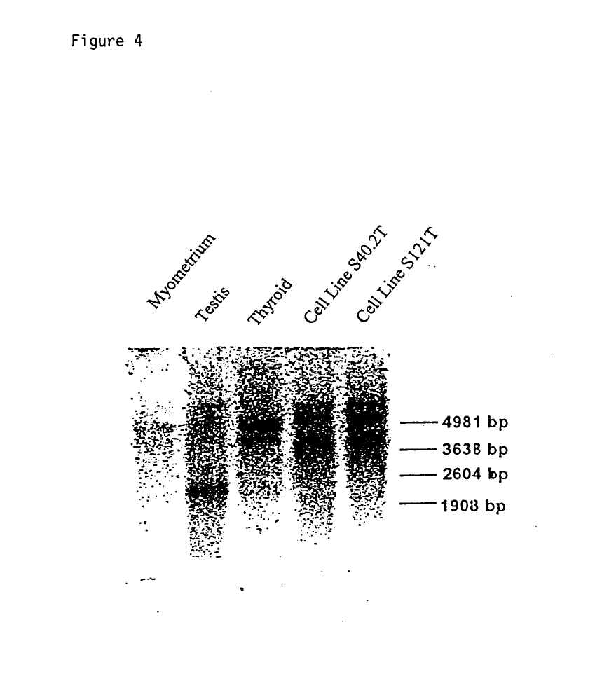

FIG. 4: shows expression studies of RITA: Northern blot analysis of poly(A)+RNA, which had been isolated from myometrium, testis, thyroid, the cell line S40.2T/SV40 and the cell line S121T/SV40 which reveals two transcripts of about 4.7 kbp and 5 kbp of different intensities in myometrium, testis and thyroid (lanes 1-3). An additional transcript of about 2.1 kbp is only found in the testis (lane 2). The cell lines S40.2T/SV40 and S121T/SV40 (lanes 4-5) express two different transcripts of approximately 5.5 kbp and 6.2 kbp. More details of the expression studies are given, among others, in example 15.

### Example 1

Cell cultures were set up from tissue specimens of follicular thyroid tumours (n=33), strumae (n=236) and of normal thyroid tissue (n=20) i.e. having the usual histological appearance and these cell cultures were processed for a chromosome analysis. The cell culture and chromosome preparation was carried out as described by Belge et al., (Belge, G., Thode, B., Bullerdiek, J., Bartnitzke, S., 1992, Cancer Genet. Cytogenet. 60, 23-26). 4 of 33 adenomas and 5 of 236 strumae had chromosome translocations involving the chromosomal band 19q13, whereas a corresponding chromosome change was not found in any specimen from the normal thyroid tissue.

### Example 2

Cell cultures of tumours with aberrations of the band 19qI3 were used to prepare cell lines for further experiments. For this a part of the SV40 genome was transfected into the cells using the calcium phosphate precipitation method as described by Kazmierczak et al. (Kazmierczak, B., Bartnitzke, S., Hartl, M., Bullerdiek, J., 1990; Cytogenet. Cell Genet. 53, 37-39). 3 cell lines were established which each contain the normal chromosome 19 and the resulting derivative chromosomes from the translocation (tab. 1)

TABLE 1
Overview of the cell lines derived from primary tumours

| Lab. Code | Material | karyotype |
|---|---|---|
| S-121 | adenoma | 46, XX, t(5; 19)(q13; q13) |
| S-141.2 | struma | 46, XX, t(2; 19)(p13; q13) |
| S-40.2 | struma | 46, XX, t(1; 19)(p35 or p36, 1; q13) |

### Example 3

Cell lines with t(5;19)(q13;q13) and t(1;19(p35-p36.1;q13) were obtained by transfection with a construct which contained the SV40 large T antigen as previously reported (Kazmierczak et al., 1990; Cytogenet Cell Genet 53:37-39); Belge et al., 1992, Cell Biol. Int. Rep., 16:339-347).

### Example 4

Fluorescence in situ hybridization (FISH) analyses were carried out after GTG band staining of the same metaphase preparations. The treatment of the metaphases and subsequent FISH experiments were carried out using the protocol of Kievits et al., (1990, Cytogenet. Cell Genet. 53:134-136). Cosmid and PAC DNA was labelled with biotin-14-dATP by nick translation (Gibco BRL, Life Technology GmbH, Eggenstein, Germany). 20 metaphases were examined for each cosmid or PAC probe. Chromosomes were counterstained with propidium iodide, analysed on a Zeiss-Axiophot fluorescence microscope using a FITC filter (Zeiss) and recorded with a power gene karyotyping system (PSI, Halladale, UK).

### Example 5

In order to obtain PAC clones which correspond to cosmid 15841 that had been isolated from the chromosome 19-specific cosmid library of the Lawrence Livermore National Laboratory (Trask et al., 1992, Genomics 14:162-167; Ashworth et al., 1995, Nat. Genet. 11:422-427), the cosmid was subcloned and sequenced on a 373 DNA sequencer (Applied Biosystems, Weiterstadt, Germany) using the primer M13 universal and M13 reverse. PAC clones were then obtained by screening a human PAC library (Genome Systems, St. Louis, USA) using a set of primers which corresponded to the sequence of cosmid 15841. PAC19/B was subcloned in three clone arrangements. As the first, EcoRI digested DNA fragments were cloned into the pGEM11zf(+) vector (Promega, Madison, USA). Two additional clone arrangements were prepared by shearing the DNA, in order to produce random fragments with an average size of 1 kbp and 3 kbp and cloned into the pTZ19R vector (MB1 Fermentas, Amherst, USA). EcoRI- and BamHI-digested DNA fragments of cosmid 15841 (Trask et al., 1991, Somat. Cell Mol. Genet. 17:117-36)f; Trask et al., 1992, loc.cit.; Ashworth et al., 1995, loc.cit.) were also cloned into the pGEMllzf(+) vector. The sequencing was carried out with standard and internal primers using an AB1 373A sequencer.

### Example 6

Alignment of the sequences and PCR primers was carried out with the Lasergene software package (DNA-Star, Madison, USA). Analyses of sequence homologies were carried out using the BLAST programme from NCB1 (Altschul et al., 1997, Nucl. Acids Res. 25:3389-3402). GENSCAN was used for gene prediction and composition (Burge and Karlin, 1997, J. Mol. Biol. 268:78-94).

### Example 7

Total RNA was isolated from two cell lines and from normal adult tissues using the TRIzol reagent (Gibco BRL, Life Technologies GmbH, Eggenstein, Germany) based on the one-step acid phenol RNA isolation method (Chomczynski and Sacchi, 1987, Anal. Biochem. 162:15-159). Poly(A)⁺−RNAs were purified by oligotex dC₁₀T₃₀ adsorption (QIAGEN, Hilden, Germany). Approximately 5 μg poly(A)⁺−RNAs were denatured and fractionated on a 1% agarose/6% formaldehyde gel and transferred to a Hybond−⁺ nylon membrane (Amersham Pharmacia, Freiburg, Germany). A partial cDNA clone (bp 17-1108) of the putative gene (RITA) (rearranged in thyroid adenomas) was used as a molecular probe. The probe was labelled with ³²P using a random primer extension protocol (Feinberg and Vogelstein, 1983, Anal. Biochem. 132:6-13). The ExpressHyb hybridization solution (Clontech Laboratories, Palo Alto, USA) was used for pre-hybridization as well as for hybridization. Pre-hybridization was carried out for 30 minutes and hybridization was carried out for one hour at 68° C. The membranes were washed twice for 20 minutes at room temperature in 2 ×SSC/0.05% SDS and twice for 20 minutes at 68° C. in 0.1×SSC/0.1% SDS. Signals were made visible using a STORM-Phosphorimager (Molecular Dynamics, Sunnyvale, USA) or by autoradiography.

### Example 8

Standard PCR amplification was carried out in a final volume of 50 μl. Approximately 60 ng template was amplified in a standard PCR reaction containing 400 nM of each primer, 300 μM dNTPs, 1 ×PCR buffer (containing 1.5 mM MgCl₂) and 2.5 U AmpliTaq (Perkin Elmer Applied Biosystems, Weiterstadt, Germany).

Alu-PCR was used to obtain genomic sequences or closely overlapping fragments (close sequencing contigs). Gene-specific primers were designed using partial sequences of RITA and sequences of PAC19/B or cosmid 15841 subclones. 400 nM of the gene-specific primer was used for an Alu-PCR reaction together with 100 nM primer TCCCAAAGTGCTGGGATTACAG and CTGCACTCCAGCCTGG. PCR products were separated on a 1.5% agarose gel, the bands of interest were cut out, purified from the gel sections using glass bead techniques (QIAExII gel isolation kit, QIAGEN, Hilden, Gennany), cloned into the pCR2.1 vector (Invitrogen, San Diego, USA) and subjected to DNA sequence analysis.

### Example 9

In order to obtain a RITA-specific cDNA probe generated by RT-PCR, total RNA from normal fibroblast primary cultures was isolated using TRIzol reagent (Gibco BRL, Life Technologies GmbH, Eggenstein, Germany). Total RNA was denatured and fractionated on a 1% agarose/6% formaldehyde gel for quality control. 5 μg of the total RNA was reversely transcribed into a first strand cDNA using M-MLV reverse transcriptase (Gibco BRL, Life Technologies GmbH, Eggenstein, Germany) using the primer AAGGATCCGTCGACATCT (17) as a start for the transcription. The extension was carried out for 45 minutes at 42° C. in a volume of 20 gl. I ill of the reaction mixture was used as a template in a standard reaction using primers ACGCAACCGCTGTGTCTCC and AGGCCTTCCCACATTCTTGAC to amplify exon 1 to exon 5 of RITA. In order to control the cDNA quality, GAPDH expression was checked with a standard RT-PCR reaction using primers GGTGAAGACGCC AGTGGACTC and GTGAAGGTCGGAGTCAACG.

### Example 10

The position of cosmid clones of the chromosome 19-specific cosmid library of the Lawrence Livermore National Laboratory, Livermore, Calif. USA relative to the breakage point of the chromosomal translocation was determined with the aid of fluorescence in situ hybridization (FISH).

In two cell lines with the translocations t(5;19) (q13;q13) and t(1;19) (p35-p36.1; q13) the chromosome 19 breakage site was located by FISH analysis between cosmid clones 15841 and 29573. These clones were isolated from the chromosome 19-specific cosmid library of the Lawrence Livermore National Laboratory (Trask et al., 1992, Genomics 14: 162-167; Ashworth et al., 1995, Nat. Genet. 11: 422-427).

### Example 11

Further tumours with aberrations of the chromosomal band 19q13 were selected from the tumour series referred to in example 1 (tab.2). Metaphase preparations of these tumours were used for fluorescence in situ hybridization. Also in this case it turned out that all breakage points were within the previously defined segment.

TABLE 2
Overview of the primary tumours used in the FISH

| Lab. Code | Material | Karyotype |
|---|---|---|
| S-172 | strumae | 46, XX, t(2; 19)(p13; q13) |
| S-216 | adenoma | 46, XX, t(10; 19)(q24; q13) |

### Example 12

A set of primers was designed corresponding to the sequence of cosmid 15841 in order to obtain PAC clones which cover the region of the chromosomal breakage sites. This set of primers was used to screen a human PAC library (Genome Systems, St. Louis, USA). The screening led to two PAC clones which are referred to herein as PAC19/A and PAC19/B. These clones were firstly used for FISH studies on the cell line S-121T/SV40 which exhibited a translocation t(5;19)(q13;q13). Signals on the normal chromosome 19 and the der(19) chromosome were detected with the PAC clone 19/A. In contrast the PAC clone 19/B exhibited signals on the normal chromosome 19, on the der(19) chromosome and on the der(5) chromosome. Hence PAC19/B spans the breakage. site of this cell line. Additional FISH studies with PAC 19/B on cells of the second cell line with the translocation t(1;19) (p35 or p36.1; q13) disclosed hybridization signals which could be mapped on the normal chromosome 19 as well as on the two derivative chromosomes which indicates that the same breakage site region was involved in this translocation. DNA sequence analysis of one of the subclones which was derived from the PAC clone PAC19/B revealed a sequence homology with several cDNA clones from the databanks. A comparison of EST sequences with the genomic sequence of the PAC clone enabled a reconstruction of the structure of the gene including a determination of its exon-intron junctions. The total size of the cDNA cloned so far is 2015 bp, but this does not necessarily represent the total length of the cDNA.

With regard to the genomic structure, the gene has 5 exons. A part of exon 5 corresponds to the 3′ non-translated region whereas its remaining part codes for a Krüppel-like zinc finger region (Schuh et al., 1986, Cell, 47: 1025-1032; Bellefroid et al., 1989, DNA, 8:377-387). Due to the high homology of exon 4 with parts of ZNF 140 (Vissing et al., 1995, FEBS, 369:153-157) and ZNF 91 (Bellefroid et al., 1993, EMBO J., 12:1363-1374) it can be concluded that this part of the gene codes for a KRAB-A domain (Bellefroid et al., 1991, Proc. Natl. Acad. Sci. USA, 88:3608-3612; Rosati et al., 1991, Nucl. Acids Res. 19: 5661-5667). Exon 3 contains a putative initiation codon which fits the consensus sequence A/GCCATGG (Kozak M. 1987, J. Mol. Biol. 196:947-950) and is why exon 1-3 cannot represent the 5=40 non-translated region. The said literature references are incorporated by way of reference.

The position of the individual exons is as follows:
exon 1: nucleotides 1-49;
exon 2: nucleotides 50-113;
exon 3: nucleotides 114-195;
exon 4: nucleotides 196-322;
exon 5: nucleotides 323-2014

The sequence coding for RITA extends from nucleotide 187-1575. A poly(A) signal is located between nucleotide 1683 and nucleotide 1689. The KRAB domain is coded by nucleotides 196-322.

A motif is understood herein as a section of a sequence at a DNA and/or protein level which is repeated in various modifications or in an identical form within the sequence. A protein domain is understood as a polypeptide chain or the part of a polypeptide chain that can independently fold into a stable tertiary structure. Zinc finger motifs may be found in the following nucleotides, as also shown in FIG. 3.
1. nucleotides 577-660;
2. nucleotides 661-744;
3. nucleotides 745-828;
4. nucleotides 829-912;
5. nucleotides 913-996;
6. nucleotides 997-1080;
7. nucleotides 1081-1164;
8. nucleotides 1165-1248;
9. nucleotides 1249-1332;
10. nucleotides 1333-1416;
11. nucleotides 1417-1500;
12. nucleotides 1591-1575.

The said zinc finger motifs are contained in the zinc finger domain.

The polypeptide shown in SEQ ID NO: 4 which is coded by the cDNA shown in SEQ ID NO: 3 has a relative molecular mass of 53,737.79 Daltons, contains 436 amino acids of which 81 are K or R (basic amino acids), 54 amino acids are D or E (acidic amino acids), 96 are A or I or L or F or W or V (hydrophobic amino acids) and 144 amino acids are N or C or Q or S or T or Y (polar amino acids). The isoelectric point is 8.9.

The transcriptional orientation of the gene is towards the telomer which can be derived from the following results. Cosmid 15841 was only mapped on the der(19) chromosome with the aid of FISH, but in both cell lines signals of PAC19/B were allocated to the der(19) chromosome as well as to the der(5) chromosome or the der(1) chromosome. On the other hand, it was shown with the aid of PCR using specific primers for individual exons that cosmid 15841 only contains the exons 3-5, whereas PAC19/B contains all so far identified exons. Hence the overlap between the two clones occurs in the 3′ part of the gene.

A typical poly(A) signal is located at bp 1684. An alternative poly(A) site AAATGAAAA is 30 bp upstream of the 3′ end.

Northern blot hybridization of a probe that is specific for the gene (bp 17-1108) on poly(A)+ Northern blots revealed a hybridization to two transcripts of approximately 4.7 kbp and 5 kbp in normal thyroid tissue and in myometrium. An additional strong band of 2.1 kbp was detected in RNA from testis. None of these transcripts was found in the two cell lines with 19 q aberrations which instead expressed two different transcripts of about 5.5 kbp and 6.2 kbp.

### Example 13

RNA was isolated by standard methods from the above-mentioned cell lines and from material preserved in liquid nitrogen of the two primary tumours with the 19q13 aberrations and this RNA was used for Northern blots. After hybridization with the cDNA of the gene RITA, clear bands were found which allow the conclusion that sequences of this PAC clone belong to a gene that is expressed in follicular thyroid tumours.

### Example 14

The methods described in examples 1 and 5 were used to carry out chromosomal analyses and molecular-cytogenetic examinations of five follicular thyroid carcinomas. Two of these tumours with complex karyotype changes also exhibited translocations with breakage points in 19q13. FISH that was also carried out yielded a location of the breakage points within the region covered by PAC (PAC19/B).

### Example 15

The fact that the gene disclosed herein and disclosed nucleic acid sequences of the target gene with the 19q13 aberrations and in particular 19q13 translocations are present in thyroid tumours is suggested by three experimental findings.

1. The breakage points are intragenic or no more than 200 kpb outside the 5′ and 3′ ends of the gene which either suggests the formation of a fusion gene or the transcriptional deregulation of the gene.

2. With regard to the expression pattern of the gene it appears that the gene is expressed in many types of normal tissue as shown by the entries in the data bank and the Northern blot results described above. Nevertheless three different transcripts have been found in normal tissue of which the smallest is only detected in testicular tissue whereas the two larger transcripts are expressed ubiquitously. In contrast a unique, unusal expression pattern for the gene RITA was only detected in two cell lines with the translocations t(5;19)(q13;q13) and t(1;19)(p35-p36.1;q13).

3. The structure of the gene suggests its involvement in transcription regulations. Its gene product, the RITA protein, belongs to the class of KRUPPEL-associated box(KRAB) zinc finger proteins (Bellefroid et al., 1991, loc.cit., Rosati et al., 1991, loc.cit.). These proteins form a large subfamily in which the KRAB-A domain has been identified as a potent transcription repressor domain (Margolin et al., 1994, Proc. Natl. Acad. Sci. USA 91:4509-4513). It has been estimated that several hundred genes which code for Krüppel-like proteins exist in the mammalian genome (Bellefroid et al., 1991, loc.cit.). Genes which code for KRAB zinc finger proteins form about a third of these proteins. The binding of the transcription repressor protein kid-1, another KRAB zinc finger protein, to an individual DNA structure has recently been demonstrated (Elser et al., 1997, J. Bio. Chem. 272:279908-27912). At first sight this suggests that RITA has a function as a transcription regulator since the 19q13 translocations lead to a loss of function. On the other hand, the lack of deletions in this region in thyroid tumours and the specificity of some of these aberrations suggest a gain of function. The Wilms' tumour gene WTI codes for a classical zinc finger protein (Call et al., 1990, Cell 60:509-520; Gessler et al., 1990, Nature 343:774-778; Haber et al., 1990, Cell 61:1257-1269) and shares some common features with the KRAB zinc finger proteins with regard to its DNA binding characteristics (Elser et al., 1997, loc.cit.). There are many experimental findings that WT1 mutations have a recessive effect with regard to the development of Wilms' tumours. In contrast a specific translocation t(11;22)(p13;q12) that is observed in desmoplastic round cell tumours leads to the formation of chimeric transcripts of the gene EWS and TWI (Ladanyi and Gerald, 1994, Cancer Res. 54:2837-2840; Rauscher et al., 1994, Cold Spring Harb. Symp. Quant. Biol. 59:137-146). In the resulting protein the RNA binding domain of EWS is replaced by the zinc finger motif of WT1. There is compelling evidence that this fusion transcript is essential for the development of desmoplastic round cell tumours which is an example of how the gene can also be impaired by a dominant gain of function.

Although the results of the Northern blot analyses suggest that the structure of RITA and its expression pattern have not yet been completely elucidated, presumably due to its close proximity to the breakage site region, the present application nevertheless discloses that RITA is a target gene for specific 19q aberrations in thyroid tumours which can be used as such for the diagnosis, prevention and treatment of functional disorders of the thyroid, hyperplasia of the thyroid and tumours of the thyroid.

The features disclosed in the previous description including the sequence protocols and in the figures and the claims can be important for the realization of the invention in its various embodiments either individually or also in any combination.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. A nucleic acid which codes for a polypeptide that influences the cellular process in thyroid tissue, wherein the polypeptide contains a KRAB domain and at least one zinc finger motif and the KRAB domain comprises the amino acid sequence of SEQ ID NO: 1 and the zinc finger motif is one which is comprised in the zinc finger domain having the amino acid sequence of SEQ ID NO: 2.

30. The nucleic acid of claim 29 comprising the nucleic acid sequence of SEQ ID NO: 3.

31. A nucleic acid coding for a polypeptide which influences the cellular process in thyroid tissue, wherein the nucleic acid hybridizes with a nucleic acid of claims 29 or 30.

32. A nucleic acid of claims 29, 30, or 31 further comprising at least one mutation.

33. A vector comprising the nucleic acid sequence of claims 29, 30, 31 or 32.

34. The vector of claim 33, further comprising at least one element which is selected from the group comprising promoters, terminators and enhancers.

35. The vector of claims 33 or 34, wherein said vector is an expression vector.

36. The vector claims 33, 34, or 35, wherein at least one promoter is in-frame with at least one part of a nucleic acid sequence according to claims 29, 30, 31 or 32 that codes for a polypeptide.

37. A polypeptide that influences the cellular process in thyroid tissue and comprises a KRAB domain and a zinc finger domain wherein the polypeptide is coded by a nucleic acid sequence according to claims 29, 30, 31 or 32.

38. The polypeptide of claim 37, wherein said polypeptide is modified.

39. A cell containing a vector of claims 33, 34, 35 or 36.

40. An antibody directed against a polypeptide according to claims 37 or 38.

41. An antibody directed against a nucleic acid according to any of claims 29, 30, 31 or 32.

42. A ribozyme directed against a nucleic acid according to claims 29, 30, 31 or 32.

43. The ribozyme of claim 42, wherein said ribozyme contains at least a part of a nucleic acid as claimed according to any of claims 29, 30, 31, or 32.

44. Use of a nucleic acid according to claims 29, 30, 31 or 32 for diagnosis and/or treatment of functional disorders of the thyroid and/or hyperplasia of the thyroid and/or thyroid tumours.

45. Use of a nucleic acid according to claims 29, 30, 31 or 32 for the manufacture of a medicament.

46. Use of a polypeptide of claims 37 or 38 for the diagnosis and/or treatment of functional disorders of the thyroid and/or hyperplasia of the thyroid and/or thyroid tumours.

47. Use of a polypeptide according to claims 37 or 38 for the manufacture of a medicament.

48. Use of an antibody according to claims 40 or 41 for diagnosis and/or treatment of functional disorders of the thyroid and/or hyperplasia of the thyroid and/or thyroid tumours.

49. Use of an antibody according to claims 40 or 41 for the manufacture of a medicament.

50. A kit for the diagnosis of functional disorders of the thyroid and/or hyperplasia of the thyroid and/or thyroid tumours comprising: at least one element which is selected from the group comprising a nucleic acid, a vector, a polypeptide, a cell, an antibody and a ribozyme, from any of claims, 29, 30, 31, 32, 33, 34, 35, 36, 37, 38, 39, 40, 41, 42 or 43.

51. Method for detecting functional disorders of the thyroid and/or hyperplasia of the thyroid and/or tumours of the thyroid, the method comprising the steps of:
contacting the thyroid materials with an agent which is selected from the group comprising a nucleic acid, a vector, a polypeptide, an antibody, a ribozyme and a cell, from any of claims 29, 30, 31, 32, 33, 34, 35, 36, 37,38, 39, 40, 41, 42 or 43, and
determining whether functional disorders of the thyroid and/or hyperplasia of the thyroid and/or tumours of the thyroid are present.

52. The method of claim 51 wherein the thyroid material is present ex vivo.

53. Use of a nucleic acid according to any of claims 29, 30, 31, or 32 as a primer.

54. A primer for preparing and/or screening and/or detecting a nucleic acid, wherein said primer is complementary to a part of a nucleic acid sequence according to claims 29, 30, 31 or 32.

55. A method for preparing a nucleic acid comprising a sequence that can be detected in thyroid tumours or strumae in which a translocation with a breakage point in the chromosomal band 19q13 is present, said sequence being located within the chromosomal band 19q13 , the method comprising the steps of:
preparing primers of claim 54 to carry out a polymerase chain reaction,
preparing a nucleic acid sequence taken from the band 19q13 of human chromosome 19 or a nucleic acid according to claims 29, 30, 31 or 32;
mixing the nucleic acid sequence or the nucleic acid with the primers; and
carrying out a polymerase chain reaction.

56. A pharmaceutical composition comprising:
at least one agent which is selected from the group comprising a nucleic acid, a vector, a polypeptide, a cell, an antibody, a ribozyme, from claims 29, 30, 31, 32, 33, 34, 35, 36, 37, 38, 39, 40, 41, 42 or 43, or combinations thereof and at least one pharmaceutically acceptable carrier.

57. Use of a nucleic acid which can hybridize with a nucleic acid according to claims 29 or 30 for the diagnosis and/or treatment of functional disorders of the thyroid and/or hyperplasia of the thyroid and/or thyroid tumours.

58. Use of a nucleic acid which can hybridize with a nucleic acid according to claims 29 or 30 for the manufacture of a medicament for the treatment of functional disorders, hyperplasia and/or tumours of the thyroid.
